(19) United States
(12) Patent Application Publication
(10) Pub. No.: US 2026/0182837 A1
(43) Pub. Date: Jul. 2, 2026

(54) Time Alignment of Physiological Signals from Multiple Devices

(71) Applicant: iRhythm Technologies, Inc., San Francisco, CA (US)

(72) Inventors: Monica C. Lin, San Francisco, CA (US); Ryan Wensley, San Francisco, CA (US); Dylan Reinsdorf, San Francisco, CA (US)

(73) Assignee: iRhythm Technologies, Inc., San Francisco, CA (US)

(21) Appl. No.: 19/436,781

(22) Filed: Dec. 30, 2025

Related U.S. Application Data
(60) Provisional application No. 63/740,281, filed on Dec. 30, 2024.

(51) Int. Cl. A61B 5/00; A61B 5/0205; A61B 5/024; A61B 5/08; A61B 5/332

(52) U.S. Cl. CPC A61B 5/0024; A61B 5/0205; A61B 5/02416; A61B 5/02438; A61B 5/08; A61B 5/332

(57) ABSTRACT

Time alignment of physiological signals for multiple devices is described. In one or more implementations, a primary device receives first physiological data from a monitoring device worn by an individual and second physiological data from an auxiliary device worn by the individual. A timing reference for the monitoring device and the auxiliary device is determined. The first physiological data and the second physiological data are aligned based on the timing reference to generate time-synchronized physiological data. The method enables accurate synchronization of physiological data from multiple wearable devices for improved analysis and diagnosis.

### RELATED APPLICATIONS

This application claims priority to U.S. Provisional Application No. 63/740,281, filed Dec. 30, 2024, and titled “Time Alignment of Physiological Signals from Multiple Devices,” which is hereby incorporated by reference in its entirety.

### BACKGROUND

Physiological monitoring systems increasingly utilize multiple wearable devices to capture different types of physiological data from different locations on a wearer's body during an observation period. By way of example, a multi-device system may include a primary monitoring device paired with one or more auxiliary devices worn on different parts of the body. However, accurately synchronizing and aligning data from multiple devices worn on different parts of the body can be challenging due to variations in sensor placement, data sampling rates, and potential time delays in data transmission or processing.

### BRIEF DESCRIPTION OF THE DRAWINGS

FIG. 1 is a block diagram of a non-limiting example of an environment that is operable to employ time alignment of physiological signals from multiple devices as described herein.

FIG. 2 depicts a non-limiting example of a monitoring device.

FIG. 3 illustrates a first example synchronization system for a time alignment process where the monitoring device acts as a primary device and the auxiliary device acts as a secondary device.

FIG. 4 illustrates a second example synchronization system for a time alignment process where the auxiliary device acts as a primary device and the monitoring device acts as a secondary device.

FIG. 5 illustrates a third example synchronization system for a time alignment process where a separate primary device coordinates synchronization between the monitoring device and the auxiliary device.

FIG. 6 illustrates an example of time alignment between ECG and PPG signals using physiological feature relationships.

FIG. 7 illustrates a method for time-aligning physiological data from multiple devices.

### DETAILED DESCRIPTION

Conventional physiological monitoring systems often utilize wearable devices to capture different types of health data from a patient. However, these systems frequently struggle with accurately synchronizing and aligning data from devices worn on different parts of the body. Variations in sensor placement, data sampling rates, and potential time delays in data transmission or processing can lead to misaligned physiological signals. This misalignment can result in various computational inefficiencies, inaccurate analysis of physiological data, and potentially incorrect medical conclusions.

Techniques for time-aligning physiological signals from multiple devices are described. In an implementation, a primary device receives physiological data from a monitoring device and an auxiliary device both worn by an individual, such as on different parts of the body. In at least one example, the monitoring device represents a chest sensor, and the auxiliary device represents a smartwatch. The primary device may determine a timing reference based on physiological data collected by the monitoring device and the auxiliary device and align the devices based on this timing reference, such as to generate time-synchronized physiological data via communication of a synchronization signal to one or more of the monitoring device or the auxiliary device.

A system for time-aligning physiological signals from multiple devices can be implemented in various configurations. For instance, either the monitoring device or the auxiliary device can serve as the primary device, with the other acting as a secondary device. Alternatively, an additional and/or separate (e.g., not body-worn) device (e.g., a smartphone and/or mobile device) can function as the primary device. In an example, the monitoring device is attached to the chest and includes an electrocardiogram (ECG) sensor, while the auxiliary device worn on an extremity and includes a photoplethysmogram (PPG) sensor.

In one or more examples, time alignment includes identification of a common physiological feature present in physiological data from the monitoring device and the auxiliary device and calculation of a time difference between instances of the physiological feature. Additionally, or alternatively, the primary device determines a relationship between different physiological features measured by the devices to perform time alignment. For example, when aligning ECG data from the chest-worn monitoring device with PPG data from the auxiliary device, the system may leverage a known relationship between heart rate (derived from the ECG data) and pulse rate (derived from the PPG data) to determine the timing reference. Alternatively, or additionally, the primary device may determine the timing reference using wireless communication methods, such as by synchronizing devices to a real-world time reference and/or computing timing offsets based on recorded timing events.

Implementations described herein may provide several advantages over conventional systems. By providing accurate time synchronization between multiple physiological signals, more comprehensive and reliable health monitoring may be provided with improved temporal resolution. As used herein, temporal resolution may refer to the precision with which timing of physiological events can be determined and correlated across multiple devices. The ability to precisely correlate events across different physiological parameters can lead to improved diagnostic accuracy, particularly for conditions in which the timing relationship between multiple physiological signals is relevant. In some applications, such as sleep apnea diagnosis, the techniques described herein may enable accurate tracking of oxygen desaturation relative to arrhythmias visible in ECG signals, which may provide contextual information about health that impacts treatment pathways. Furthermore, the flexibility in device configuration and synchronization methods described herein enable the techniques described herein to be adapted to a range of monitoring scenarios and device types, enhancing practical applicability in various healthcare settings.

The techniques described herein provide a technical solution for time-aligning physiological signals from multiple wearable devices in the field of physiological monitoring. The method utilizes wearable devices including a monitoring device and an auxiliary device that measure physiological parameters from a body of an individual. These devices include sensors such as electrodes for ECG, electromyography (EMG), and/or bioimpedance (BioZ) measurement; optical sensors for PPG measurement; accelerometers; temperature and/or heat flux sensors; and/or respiration sensors that detect and measure physiological phenomena as electrical signals. The monitoring device and the auxiliary device generate time-stamped physiological data that is transmitted wirelessly or via wired connections to a primary device for processing.

In one or more implementations, the primary device performs real-time processing of physiological data, as the data is received from the monitoring device and the auxiliary device. The synchronization algorithm may analyze incoming physiological data streams in real-time to detect timing discrepancies between devices and generate synchronization signals during an ongoing observation period. This real-time processing enables the primary device to continuously or semi-continuously (e.g., at a predetermined frequency or when triggered by passing some preset threshold) monitor timing alignment and transmit updated synchronization signals to correct for clock drift or timing errors as they occur. By way of example, the primary device may receive ECG data from the monitoring device and PPG data from the auxiliary device in real-time, extract respiratory patterns from both signals as the data arrives, and calculate a timing offset on-the-fly to maintain accurate synchronization throughout the observation period. This real-time computational processing operates on streaming physiological data and generates timing corrections within seconds or sub-seconds of detecting timing discrepancies, for example. In one or more variations, the physiological data may be processed after the data collection period.

The techniques described herein may adjust how the monitoring device and the auxiliary device operate. By way of example, the synchronization signal generated by the primary device may cause the monitoring device and/or the auxiliary device to adjust internal clocks, modify timestamp generation, and/or alter data collection timing. This represents a change to the operation of the devices that improves their technical functioning by enabling accurate temporal correlation of physiological measurements. The time-synchronized physiological data generated through this process provides a technical improvement over conventional multi-device monitoring systems that lack accurate synchronization, enabling more precise correlation of physiological events. Accordingly, the techniques described herein improve the functioning of multi-device monitoring systems by enabling accurate temporal correlation of physiological events, such as correlating oxygen desaturation events measured at a finger with cardiac arrhythmias detected at the chest. This temporal correlation enables improved diagnostic capabilities for conditions where timing relationships between physiological parameters may be relevant.

In some aspects, the techniques described herein relate to a method for time-alignment between physiological signals from a monitoring device and an auxiliary device, including: receiving, by a primary device, first physiological data from the monitoring device worn by an individual; receiving, by the primary device, second physiological data from the auxiliary device worn by the individual; determining a timing reference for the monitoring device and the auxiliary device; and aligning the first physiological data and the second physiological data based on the timing reference to generate time-synchronized physiological data.

In some aspects, the techniques described herein relate to a method, wherein aligning the first physiological data and the second physiological data based on the timing reference includes generating, by the primary device, a synchronization signal for transmission to at least one of the monitoring device or the auxiliary device based on the timing reference.

In some aspects, the techniques described herein relate to a method, wherein the timing reference is a real-world time reference, and wherein the synchronization signal includes timing information that instructs at least one of the monitoring device or the auxiliary device to adjust an internal clock relative to the real-world time reference.

In some aspects, the techniques described herein relate to a method, further including: recording, by at least one of the monitoring device or the auxiliary device, timing information included in the synchronization signal relative to internal timing information.

In some aspects, the techniques described herein relate to a method, wherein the monitoring device is the primary device, and the auxiliary device is a secondary device.

In some aspects, the techniques described herein relate to a method, wherein the auxiliary device is the primary device, and the monitoring device is a secondary device.

In some aspects, the techniques described herein relate to a method, wherein the primary device is an additional auxiliary device.

In some aspects, the techniques described herein relate to a method, wherein determining the timing reference includes: identifying a common physiological feature in the first physiological data and the second physiological data; and calculating, as the timing reference, a time difference between occurrences of the common physiological feature in the first physiological data and the second physiological data.

In some aspects, the techniques described herein relate to a method, further including: identifying a first physiological feature in the first physiological data; identifying a second physiological feature in the second physiological data; and determining the timing reference based at least in part on a relationship between the first physiological feature and the second physiological feature.

In some aspects, the techniques described herein relate to a method, wherein the first physiological feature is heart rate, and the second physiological feature is pulse rate.

In some aspects, the techniques described herein relate to a method, wherein: the monitoring device is configured to be attached to a chest of the individual and includes an electrocardiogram (ECG) sensor for measuring, as the first physiological data, ECG data; and the auxiliary device is configured to be attached to an extremity of the individual and includes a photoplethysmogram (PPG) sensor for measuring, as the second physiological data, PPG data.

In some aspects, the techniques described herein relate to a method, wherein the timing reference is based at least in part on respiratory signals extracted from the ECG data and the PPG data.

In some aspects, the techniques described herein relate to a system for physiological monitoring of an individual, including: a monitoring device configured to measure first physiological data at a first body location of the individual; an auxiliary device configured to measure second physiological data at a second body location of the individual; and a synchronization algorithm implemented as computer-readable instructions stored in a non-transitory memory that, when executed by a processing device, cause the processing device to perform operations including: determining a timing reference for the monitoring device and the auxiliary device; and time-aligning the first physiological data and the second physiological data based on the timing reference to generate time-synchronized physiological data.

In some aspects, the techniques described herein relate to a system, wherein the monitoring device or the auxiliary device includes the processing device.

In some aspects, the techniques described herein relate to a system, further including the processing device, and wherein the processing device is a smartphone, gateway or laptop.

In some aspects, the techniques described herein relate to a system, wherein to determine the timing reference, the operations further include: identifying a common physiological feature in the first physiological data and the second physiological data; and calculating, as the timing reference, a time difference between occurrences of the common physiological feature in the first physiological data and the second physiological data.

In some aspects, the techniques described herein relate to a system, wherein to determine the timing reference, the operations further include: transmitting timing information corresponding to a real-world time to at least one of the monitoring device or the auxiliary device; receiving recorded timing events from at least one of the monitoring device and the auxiliary device, each recorded timing event including the timing information and a corresponding local timestamp; and determining the timing reference based on the timing information and the corresponding local timestamp.

In some aspects, the techniques described herein relate to a method for time-alignment between physiological signals from multiple wearable devices, including: measuring, by a monitoring device, electrocardiogram (ECG) data at a first body location of an individual; measuring, by an auxiliary device, photoplethysmogram (PPG) data at a second body location of the individual; transmitting, by a primary device, a synchronization signal including a timing reference to at least one of the monitoring device or the auxiliary device; and time-aligning, by the primary device, the ECG data and the PPG data based on the timing reference.

In some aspects, the techniques described herein relate to a method, further including: identifying at least one physiological feature in the ECG data and the PPG data; and determining the timing reference based on a time difference between occurrences of the at least one physiological feature in the ECG data and the PPG data.

In some aspects, the techniques described herein relate to a method, wherein the timing reference corresponds to a real-world time.

It should be understood that many variations are possible based on the disclosure herein. Although features and elements are described above in particular combinations, each feature or element is usable alone without the other features and elements or in various combinations with or without other features and elements.

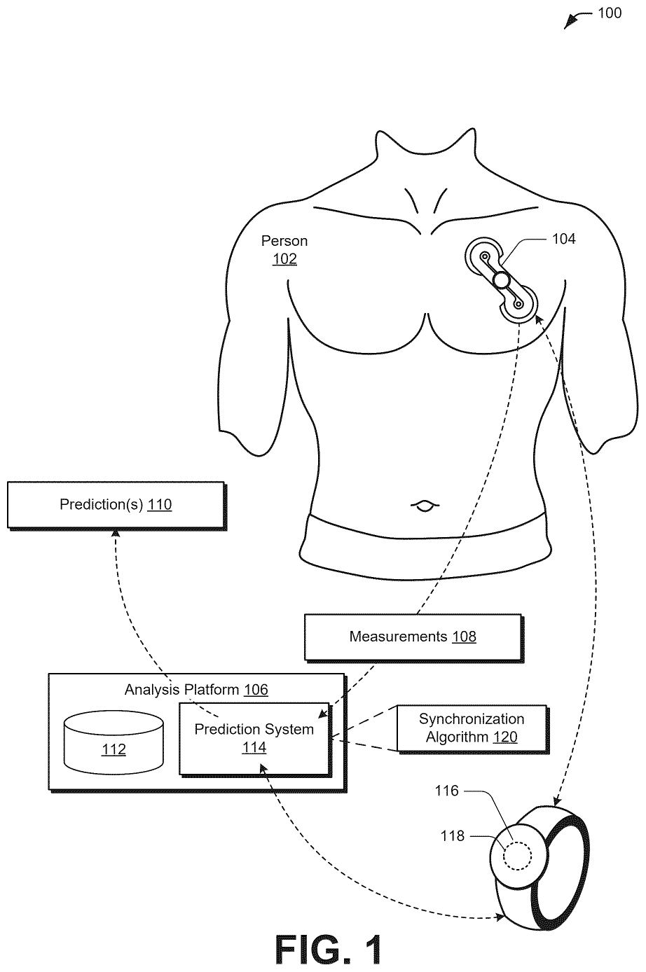

FIG. 1 is a block diagram of a non-limiting example 100 of an environment that is operable to employ time alignment of physiological signals from multiple devices as described herein. The illustrated example 100 includes a monitored subject, e.g., a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. Alternatively, or in addition, the monitoring device 104 may be used to output the measurements 108 (e.g., a time sequence of measurements such as a time sequence of electric potential measurements), which may indicate an observation or be used to generate an assessment, diagnosis, or prediction of one or more events.

In connection with the monitoring device 104, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

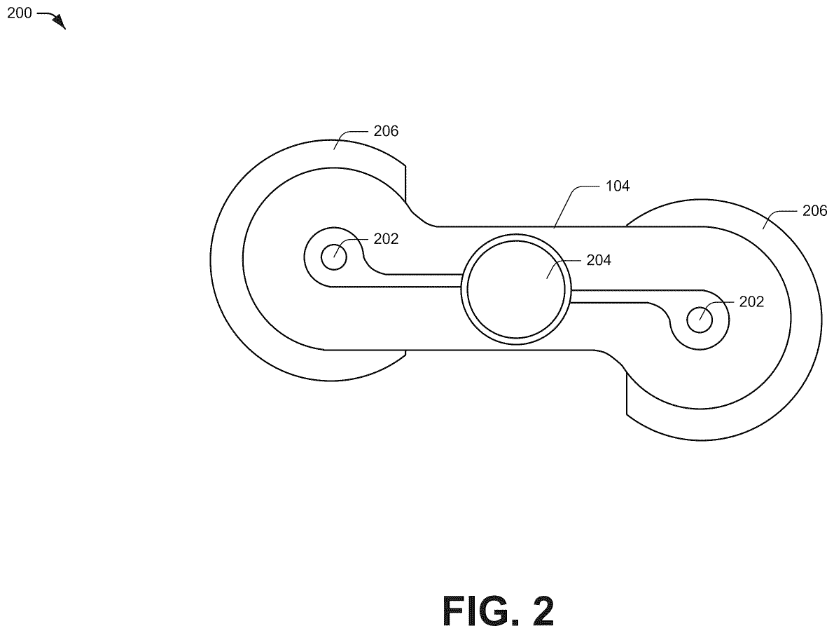

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements.

In one or more implementations, the monitoring device 104 may be configured to offload measurements and/or other data from the monitoring device during the course of the observation period. By way of example, the monitoring device 104 may offload the measurements by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea.

To the extent that the monitoring device 104 may be configured to store the measurements 108 for an entirety of an observation period, in one or more implementations, the monitoring device 104 may be configured without wireless transmission means, e.g., without any antennas to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate one or more predictions 110. For example, the measurements 108 (and/or other measurements such as accelerometer data and oxygen saturation (SpO2) measurements) may be processed by a smartphone associated with a user, a smartphone or other dedicated device associated with the monitoring device 104, and/or one or more server computers at a data center or other location that can be utilized by an entity associated with the monitoring device 104, to name just a few. In other words, those other devices may implement at least a portion of the prediction system 114.

In one or more implementations, the monitoring device 104 is configured to transmit the measurements 108 to a separate device over a wired connection with the separate device, e.g., via USB-C or some other physical, communicative coupling. As used herein, a “separate device” is meant to denote a device that is not body-worn. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the separate device. Such a connection may be used in scenarios where the monitoring device 104 is mailed by the person 102 after the observation period, such as to a health care provider, telemedicine service, provider of the monitoring device 104, or medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with additional (e.g., separate) devices using one or more wireless communication protocols or techniques. By way of example, the monitoring device 104 may communicate with the additional devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennas and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone or other gateway device associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc. As noted above, examples of such additional measurements include but are not limited to accelerometer data and/or oxygen saturation (e.g., SpO2) measurements.

In the illustrated example 100, the analysis platform includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that, in variations, the prediction system 114 may output different combinations of multiple predictions.

In at least one implementation, the prediction system 114 uses machine learning to generate at least a portion of the one or more predictions 110. The one or more predictions 110 may include, for example, predictions related to sleep apnea, cardiac arrhythmias, or other conditions where timing relationships between physiological signals may be relevant. In some cases, the one or more predictions 110 may include predictions about oxygen desaturation events relative to cardiac events visible in ECG signals, which may provide contextual information about health that impacts treatment pathways. In one or more implementations, the one or more predictions 110 may include an assessment or diagnosis related to a health condition or disease state. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the measurements 108, such as to extract various cardiovascular and/or other features from the sequences of measurements. In the illustrated example 100, for instance, the one or more predictions 110 correspond to the output of the prediction system 114.

The illustrated example 100 further includes an auxiliary device 116. The auxiliary device 116 is configured to implement the techniques described herein for time alignment of physiological signals from multiple devices. In various examples, the auxiliary device 116 is configured as a wearable device that complements the monitoring device 104 in collecting physiological data from the person 102. In some aspects, the auxiliary device 116 may be designed to be worn on a different part of the body than the monitoring device 104, such as on a wrist, finger, ankle, or neck. The auxiliary device 116 may include one or more sensors 118 configured to capture different types of physiological measurements. In the illustrated example 100, the one or more sensors 118 are shown as a dashed outline to indicate that the one or more sensors 118 may be positioned on a back surface of the auxiliary device 116. In implementations where the auxiliary device 116 is configured as a wrist-worn device, for example, the one or more sensors 118 may be disposed on a skin-contacting surface that faces inward toward the wrist when worn. The positioning of the one or more sensors 118 on the back surface may enable the sensors to detect physiological signals through contact with or proximity to skin, such as optical signals for photoplethysmography (PPG) measurements, electrical signals (e.g., electrocardiography (ECG), electromyography (EMG), and/or bioimpedance), temperature measurements, or other physiological parameters.

The one or more sensors 118 may be the same type of sensor or a different type of sensor as those included in the monitoring device 104. By way of example, the one or more sensors 118 may include PPG sensors, respiration sensors, accelerometers, temperature sensors, electrodes, and so forth. As a non-limiting, illustrative first example, the one or more sensors 118 include a respiration sensor, and the monitoring device 104 also includes a respiration sensor, thus enabling time alignment through common physiological features. In a non-limiting, illustrative second example, the one or more sensors 118 include a different type of sensor than the monitoring device 104 (e.g., the monitoring device 104 has an ECG sensor, while the auxiliary device 116 has a PPG sensor), thus enabling time alignment through related physiological features.

The auxiliary device 116 may capture physiological measurements that include, but are not limited to, heart rate, blood oxygen saturation, skin temperature, motion data, PPG data, respiration data, and the like. In one or more implementations, the auxiliary device 116 may be configured as a neck-worn device that includes a respiration sensor for measuring respiratory activity. In another example, the auxiliary device 116 may be configured as a finger-worn ring device that measures SpO2 via PPG waveforms. In yet another example, the auxiliary device 116 may be configured as a smartwatch worn on a wrist. This is by way of example and not limitation, and a variety of properties and form factors of the auxiliary device 116 are considered.

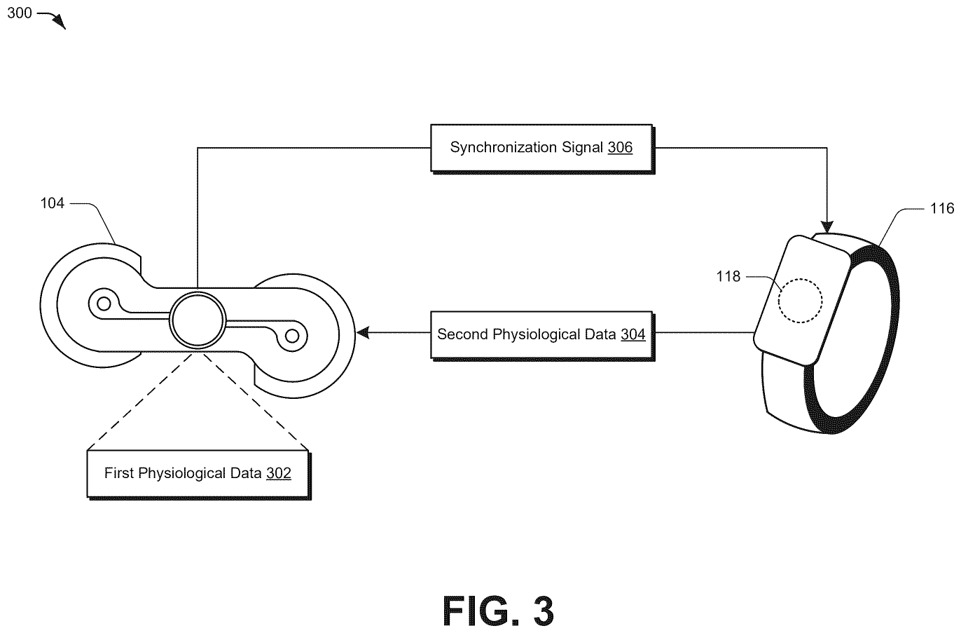

For instance, the auxiliary device 116 may share one or more properties with the monitoring device 104 as described above and below in more detail. The auxiliary device 116, for instance, may be configured to communicate with the analysis platform 106 to perform a variety of functionality. In one or more examples, the auxiliary device 116 is configured to offload measurements during an observation period, such as through wired or wireless connections to an external computing device. The auxiliary device 116 may also employ data compression techniques to reduce battery usage and facilitate extended wear. Additionally, the auxiliary device 116 may be designed with various communication capabilities, allowing it to transmit data directly to the analysis platform 106 or to other devices such as smartphones, tablets, gateways, or laptops. In some cases, the auxiliary device 116 serves as a primary device, coordinating data collection and synchronization with the monitoring device 104 and other potential sensors, as described in more detail below with respect to FIGS. 3-5. In at least one variation, a separate device such as a smartphone, a gateway, or a laptop may serve as the primary device, coordinating synchronization between the monitoring device 104 and one or more auxiliary devices 116. In at least one other variation, the monitoring device 104 serves as the primary device to coordinate data collection and synchronization with the auxiliary device 116. The monitoring device 104 and the auxiliary device 116 may also support configurations with multiple auxiliary devices worn simultaneously, such as a chest-worn monitoring device 104 paired with both a neck-worn respiration sensor and a finger-worn ring device, enabling comprehensive multi-parameter physiological monitoring with accurate time alignment across a plurality of devices.

In the illustrated example 100, the analysis platform 106 includes a synchronization algorithm 120. The synchronization algorithm 120 represents functionality of the prediction system 114 to determine timing references for physiological data obtained from multiple devices and/or to generate synchronization signals for aligning data collection timing. In one or more implementations, the synchronization algorithm 120 may determine the timing reference by identifying a common physiological feature present in physiological data from the monitoring device 104 and the auxiliary device 116 and calculating a time difference between occurrences of the common physiological feature. As used herein, a common physiological feature may refer to a physiological parameter or signal that is measured by multiple devices and/or derived from measurements obtained by multiple devices and that exhibits corresponding patterns or characteristics across those devices. By way of example, the sensors on the monitoring device 104 and the one or more sensors 118 on the auxiliary device 116 may measure a time-varying signal that can be compared between devices. Examples of physiological features that may be measured include respiration, heart rate, pulse rate, and so forth. In an implementation where respiratory sensors are included on multiple devices, the respiratory signals received from those devices can be utilized to determine a delay (e.g., a time offset) between the signals, and the synchronization algorithm 120 may correct for the delay by adjusting measurement timestamps and/or generating synchronization signals based on the calculated time difference.

Alternatively, or in addition, the synchronization algorithm 120 may utilize wireless communication methods to achieve time alignment, such as by coordinating timing information between the monitoring device 104 and the auxiliary device 116 or through a separate device that is not body-worn. The synchronization algorithm 120 may further generate one or more synchronization signals for transmission to the monitoring device 104 and/or the auxiliary device 116 to align data collection timing, which may enable the prediction system 114 to process time-synchronized physiological data for improved diagnostic accuracy. In one or more implementations, the synchronization algorithm 120 may use a real-world time reference for the time alignment.

It is to be appreciated that although the analysis platform 106 is shown separately from the monitoring device 104 and the auxiliary device 116, in one or more implementations, the analysis platform 106 may be implemented in whole or in part at the monitoring device 104. Alternatively, or in addition, the analysis platform 106 may be implemented in whole or in part at the auxiliary device 116. In other implementations, the analysis platform 106, or portions thereof (e.g., the synchronization algorithm 120) may be implemented using one or more computing devices external to the monitoring device 104 and the auxiliary device 116, such as one or more computing devices associated with the person 102 (e.g., a mobile phone, tablet device, laptop, desktop, or smartwatch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices. The synchronization algorithm 120 may similarly be implemented in whole or in part at the monitoring device 104, the auxiliary device 116, or a separate device, enabling the configurations shown in FIGS. 3-5 and discussed below where different devices may act as the primary device coordinating time alignment. The primary device, for instance, may serve as a processing device for executing the synchronization algorithm 120 based on computer-readable instructions stored in a non-transitory memory.

In this way, the illustrated example 100 enables time alignment of physiological signals from multiple devices worn on different parts of the body. The analysis platform 106, through the synchronization algorithm 120, may coordinate data collection from the monitoring device 104 and the auxiliary device 116 to achieve accurate temporal correlation between different physiological measurements. This approach may provide flexibility in device configurations, as the primary device coordinating synchronization may be implemented as the monitoring device 104, the auxiliary device 116, or a separate device such as a smartphone or gateway. By enabling precise time alignment across multiple measurement modalities, the illustrated example 100 may support comprehensive physiological monitoring and improved diagnostic accuracy for conditions where timing relationships between physiological signals may be relevant.

FIG. 2 depicts a non-limiting example 200 of a monitoring device. The illustrated example 200 depicts the monitoring device 104. As described above in more detail, in various examples the auxiliary device 116 includes one or more properties and/or features of the monitoring device 104. The auxiliary device 116 may similarly include one or more sensors 118 as described above, which may be the same type or a different type as the one or more sensors 202 of the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on communications with the one or more sensors 202 that are indicative of some aspect of the person 102, such as the person 102's heart's electrical activity. In one or more implementations, the processor further generates one or more communicable packages of data that include one or more of the measurements 108 and/or other measurements, such as accelerometer data and oxygen saturation (SpO2) measurements. Alternatively, or additionally, the processor produces and/or causes storage of other data, which may be used for time-aligning physiological data from multiple devices.

In implementations where the monitoring device 104 is configured for wireless transmission, the transmitter 204 may transmit the measurements wirelessly as a stream of data to a computing device. In one or more implementations, for instance, the monitoring device 104 is configured to transfer (e.g., transmit and/or receive) information (e.g., electrical potential measurements) via a Bluetooth® Low Energy (BLE) connection. Alternatively, or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

FIG. 3 illustrates a first example synchronization system 300 for a time alignment process where the monitoring device 104 acts as a primary device and the auxiliary device 116 acts as a secondary device. A primary device, for instance, may refer to a device that coordinates data collection and synchronization between multiple physiological monitoring devices, whereas a secondary device may refer to a device that receives synchronization signals and timing information from the primary device to align its data collection with other devices in the system.

In the first example synchronization system 300, the monitoring device 104 may be attached to the chest of the person 102, while the auxiliary device 116 may be worn on an extremity of the person 102, such as a wrist or finger, or on a neck. As non-limiting examples, the auxiliary device 116 may be a smartwatch worn on the wrist, a finger ring device measuring SpO2 and/or PPG, or a neck-worn device measuring respiration. Further, while not depicted, in some examples, more than one auxiliary device 116 may be incorporated. The monitoring device 104, acting as the primary device, may coordinate synchronization with multiple auxiliary devices 116, which may each measure different physiological parameters in some examples. For instance, the monitoring device 104 may be a chest patch paired with both a first auxiliary device 116 comprising a neck-worn respiration sensor and a second auxiliary device 116 comprising a finger ring device measuring SpO2 and/or PPG, enabling comprehensive multi-parameter physiological monitoring.

The monitoring device 104 may collect first physiological data 302 (e.g., as the measurements 108), which can include one or more of a variety of measurements such as electrocardiogram (ECG) data, heart rate, heart rate variability, respiration rate, skin temperature, motion data from an accelerometer, and so forth. The auxiliary device 116 may simultaneously collect second physiological data 304 using the one or more sensors 118. In some cases, the first physiological data 302 and the second physiological data 304 may overlap with respect to the types of measurements collected. By way of example, the first physiological data 302 and the second physiological data 304 may pertain to a common physiological feature. Alternatively, or additionally, the first physiological data 302 and the second physiological data 304 include different types of measurements, e.g., a first physiological feature (e.g., ECG measurements) and a second physiological feature (e.g., PPG measurements).

When the monitoring device 104 is configured as the primary device, the monitoring device 104 is configured to receive the second physiological data 304 from the auxiliary device 116, such as through one or more wireless communication modalities, e.g., Bluetooth®, Wi-Fi®, and so forth. In this example, the monitoring device 104 determines a timing reference for the monitoring device 104 and the auxiliary device 116. To do so, in some implementations the monitoring device 104 may analyze the collected data (e.g., via the synchronization algorithm 120) to identify common physiological events or patterns present in both datasets. For example, if both the monitoring device 104 and the auxiliary device 116 include respiration sensors, respiratory signals received from each can be utilized (e.g., by the synchronization algorithm 120) to determine a delay between the signals that may be used as the timing reference. Alternatively, a single physiological signal such as respiration can be derived from two different sensors on the monitoring device 104 and the auxiliary device 116 (e.g., respiration may be derived from ECG on the monitoring device 104 and from PPG on the auxiliary device 116). Alternatively, the monitoring device 104 may utilize the synchronization algorithm 120 to identify relationships between different physiological features, such as matching a heart rate calculated from ECG measurements with a pulse rate calculated from PPG measurements to determine the timing reference. In yet another example, alternatively or in addition, the monitoring device 104 (e.g., via the synchronization algorithm 120) may detect timestamps in the first physiological data 302 and the second physiological data 304. By comparing timestamps and/or a relative timing of physiological events, the monitoring device 104 may calculate a time difference between the two datasets, which may be used as the timing reference. Accordingly, the timing reference may be determined using various techniques, such as cross-correlation analysis, peak detection algorithms, or machine learning models trained to recognize temporal relationships between different physiological signals. Once the timing reference is determined, the monitoring device 104 may use this information to generate a synchronization signal 306, which may be transmitted to the auxiliary device 116 to align data collection timing with that of the monitoring device 104, as described in more detail below.

For instance, to achieve time alignment between the monitoring device 104 and the auxiliary device 116, the synchronization signal 306 may be transmitted from the monitoring device 104 to the auxiliary device 116. In some cases, the synchronization signal 306 may include timing information that causes the auxiliary device 116 to adjust an internal clock and/or timestamp data relative to the monitoring device 104. Alternatively, the auxiliary device 116 records the timing information received from the monitoring device 104 relative to its internal timing information for use in post-processing (e.g., after a data collection period) to time-align the signals from the monitoring device 104 and the auxiliary device 116. By way of example, the timing information received from the monitoring device 104 may be used to determine a time offset relative to the internal timing information of the auxiliary device 116, and the time offset may be used in the post-processing to time-align the second physiological data 304 with the first physiological data 302. The monitoring device 104, acting as the primary device, may transmit the synchronization signal 306 with timing information that the auxiliary device 116, acting as the secondary device, receives and records. This synchronization process allows for precise temporal correlation between the first physiological data 302 and the second physiological data 304. In this way, the techniques described herein allow for time alignment to be achieved with temporal resolution that may be suitable for accurately correlating physiological events across different measurement types. By way of example, second or sub-second resolution may be achieved.

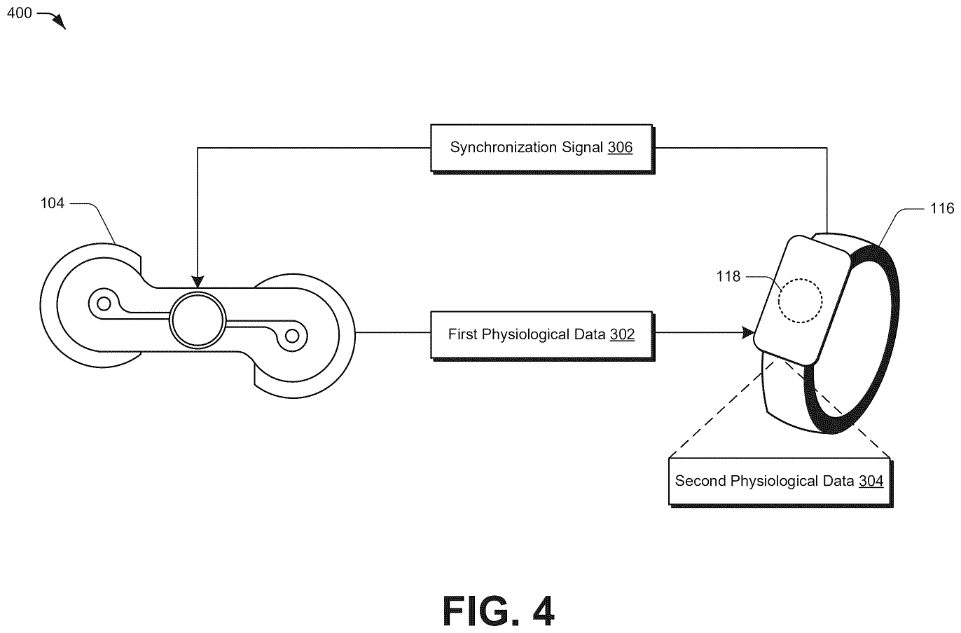

FIG. 4 illustrates a second example synchronization system 400 for a time alignment process where the auxiliary device 116 acts as a primary device and the monitoring device 104 acts as a secondary device. In this configuration, the auxiliary device 116 receives the first physiological data 302 from the monitoring device 104. The auxiliary device 116 processes (e.g., via the synchronization algorithm 120) the first physiological data 302 received from the monitoring device 104 and the second physiological data 304 collected by the one or more sensors 118 to determine the timing reference for the two data sets. The auxiliary device 116 generates the synchronization signal 306 and transmits the synchronization signal 306 to the monitoring device 104 to align data collection timing, such as described above with respect to the monitoring device 104 as the primary device in the first example synchronization system 300 of FIG. 3. It is to be appreciated that the auxiliary device 116 may transmit the synchronization signal 306 to a plurality of other auxiliary devices in addition to the monitoring device 104, such as in examples including more than two devices. The second example synchronization system 400 demonstrates the flexibility of the techniques described herein, where the monitoring device 104 or the auxiliary device 116 may serve as the primary device for coordinating time alignment between multiple physiological monitoring devices.

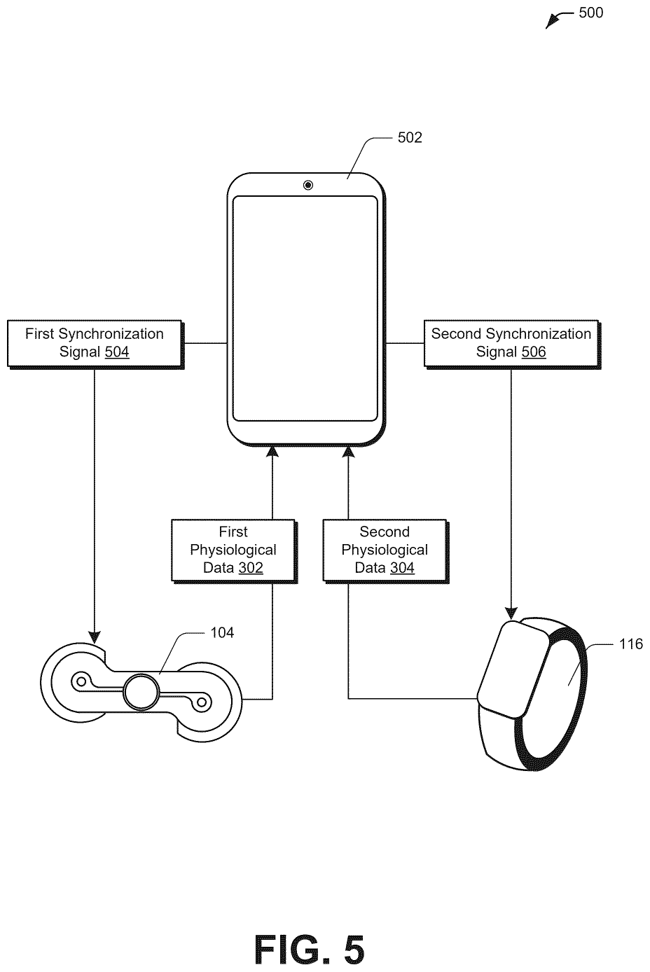

FIG. 5 illustrates a third example synchronization system 500 for a time alignment process where a separate primary device 502 coordinates synchronization between the monitoring device 104 and the auxiliary device 116. In this configuration, the separate primary device 502 may be an additional auxiliary device such as a smartphone, gateway, laptop, or another computing device capable of coordinating multiple body-worn sensors.

The separate primary device 502 may receive the first physiological data 302 from the monitoring device 104 and the second physiological data 304 from the auxiliary device 116. As described above, the first physiological data 302 and the second physiological data 304 can measure a variety of physiological features. The auxiliary device 116 may collect the second physiological data 304 using the one or more sensors 118, which may be the same type or a different type as the one or more sensors 202 of the monitoring device 104. The monitoring device 104 and the auxiliary device 116 may transmit the first physiological data 302 and the second physiological data 304, respectively, to the separate primary device 502 using various wireless communication protocols, such as Bluetooth®, Wi-Fi®, and the like.

To achieve time alignment between the monitoring device 104 and the auxiliary device 116, the separate primary device 502 may generate and transmit one or more synchronization signals. For instance, the separate primary device 502 may send a first synchronization signal 504 to the monitoring device 104 and a second synchronization signal 506 to the auxiliary device 116. The first synchronization signal 504 and the second synchronization signal 506 may contain timing information that allows the monitoring device 104 and the auxiliary device 116 to adjust their internal clocks or timestamp their data relative to a common time reference. Alternatively, the monitoring device 104 may record the timing information received in the first synchronization signal 504 relative to its internal timing information for use in post-processing time correction (e.g., after a data collection period), and/or the auxiliary device 116 may record the timing information received in the second synchronization signal 506 relative to its internal timing information for use in the post-processing time correction, such as described above with respect to FIG. 3. The common time reference may be global real-time, such as Coordinated Universal Time (UTC) or Greenwich Mean Time (GMT), for instance. The common time reference may provide the timing reference, for example. In implementations where the separate primary device 502 is connected to a cellular network or other time reference source, the separate primary device 502 may obtain accurate timing information and distribute the timing information to the monitoring device 104 and the auxiliary device 116 via the first synchronization signal 504 and the second synchronization signal 506, respectively. The monitoring device 104 and the auxiliary device 116 may be configured to sync to the common time reference with minimal time drift during a data collection period (e.g., an observation period). In some cases, an initial time-syncing event may occur at the beginning of an applicable period, such as upon activation of the devices and/or the beginning of the data collection period. This initial synchronization may establish a baseline for subsequent time alignment throughout the data collection period. Real-time clocks within the monitoring device 104 and the auxiliary device 116 may maintain time alignment during the data collection period with sufficient accuracy for correlating physiological events (e.g., time-alignment within seconds or sub-seconds).

Alternatively, or in addition to clock-based synchronization, the separate primary device 502 may determine, as the timing reference, a time offset between the first physiological data 302 and the second physiological data 304 by analyzing the physiological data itself (e.g., using the synchronization algorithm 120). In implementations where the monitoring device 104 and the auxiliary device 116 include sensors that measure a common physiological feature, the separate primary device 502 may identify the common physiological feature in the first physiological data 302 and the second physiological data 304 and calculate a time difference between occurrences of the common physiological feature. For example, if both the monitoring device 104 and the auxiliary device 116 include respiration sensors, the separate primary device 502 may analyze respiratory signals from both devices to determine a delay (e.g., the time offset) between the signals. The separate primary device 502 may then generate the first synchronization signal 504 and the second synchronization signal 506 based on the calculated time difference to correct for the delay.

In at least one variation, the separate primary device 502 may determine the timing reference based on a relationship between different physiological features measured by the monitoring device 104 and the auxiliary device 116. For instance, where the monitoring device 104 measures ECG data and the auxiliary device 116 measures PPG data, the separate primary device 502 may leverage a relationship between a heart rate calculated from the ECG data and a pulse rate calculated from the PPG data to determine the timing reference. By way of example, using the synchronization algorithm 120, the separate primary device 502 may identify corresponding cardiac events in the first physiological data 302 and the second physiological data 304 and account for a physiological delay between, e.g., cardiac electrical activity and peripheral pulse to generate the first synchronization signal 504 and the second synchronization signal 506. Alternatively, or in addition, the synchronization algorithm 120 may process the ECG data to determine an ECG-derived respiratory rate and process the PPG data to determine a PPG-derived respiratory rate. The synchronization algorithm 120 may determine the timing reference based on the ECG-derived respiratory rate and the PPG-derived respiratory rate.

Accordingly, the separate primary device 502 may use the first physiological data 302 received from the monitoring device 104 and the second physiological data 304 received from the auxiliary device 116, along with timing information from the first synchronization signal 504 and the second synchronization signal 506, in a variety of ways to align the data from both devices. This alignment process allows for precise temporal correlation between physiological measurements collected by the monitoring device 104 and the auxiliary device 116. Further, by utilizing the separate primary device 502 for time alignment, the third example synchronization system 500 may provide flexibility in coordinating multiple physiological monitoring devices. This approach enables accurate synchronization of data from various sources, which supports analysis of complex physiological events that include multiple parameters. For example, the separate primary device 502 may coordinate time alignment between a chest-worn monitoring device 104 and multiple auxiliary devices 116, such as a neck-worn respiration sensor and a finger ring device measuring SpO2 and/or PPG, enabling a comprehensive assessment of conditions where timing relationships between oxygen desaturation, respiratory events, and cardiac arrhythmias may be relevant.

By precisely synchronizing data from multiple devices, the first example synchronization system 300, the second example synchronization system 400, and the third example synchronization system 500 may enable accurate correlation of physiological events across different measurement modalities. This may be valuable for use in diagnosing conditions where timing relationships between various physiological signals may be relevant, such as sleep apnea or certain cardiac arrhythmias. Additionally, the flexibility of the first example synchronization system 300, the second example synchronization system 400, and the third example synchronization system 500 in accommodating multiple devices may allow for a more comprehensive physiological assessment, potentially leading to improved diagnostic accuracy and patient care.

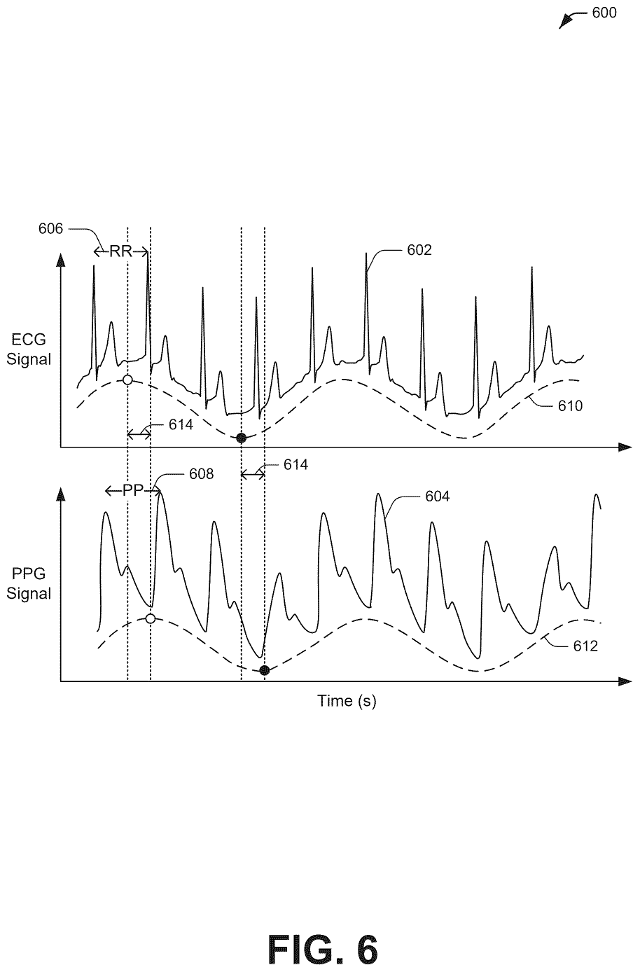

FIG. 6 illustrates an example 600 of time alignment between ECG and PPG signals using physiological feature relationships. The example 600 includes an ECG signal 602 and a PPG signal 604, both shown with respect to time. By way of example, the ECG signal 602 and the PPG signal 604 may be time-aligned according to time stamps generated by respective measurement devices. The ECG signal 602, for instance, may be measured by the monitoring device 104 (e.g., via the one or more sensors 202), and the PPG signal 604 may be measured by the auxiliary device 116 (e.g., via the one or more sensors 118). In at least one variation, the PPG signal 604 is measured by the monitoring device 104, and the ECG signal 602 is measured by the auxiliary device 116.

The ECG signal 602 is shown with characteristic waveforms including R-peaks that represent ventricular depolarization. An RR interval 606 is indicated between two consecutive R-peaks in the ECG signal 602, representing a time between successive heartbeats. A heart rate may be calculated from the RR interval 606, such as by dividing sixty seconds by the RR interval 606 measured in seconds to obtain the heart rate in beats per minute. The PPG signal 604 is depicted with pulse waveforms that correspond to blood volume changes in peripheral vasculature. A PP interval 608 is indicated between two corresponding peaks in the PPG signal 604, representing the time between successive pulse waves. A pulse rate may be calculated from the PP interval 608 in a similar manner. The ECG signal 602 represents electrical timing of cardiac events and may be more stable for timing purposes, while the PPG signal 604 represents mechanical timing of peripheral blood flow. Although the PPG signal 604 may be subject to variability from vasoconstriction, sensor pressure changes, waveform distortions, and motion artifacts, the ECG-derived heart rate and the PPG-derived pulse rate may provide a reliable time-syncing metric. Although the ECG signal 602 and the PPG signal 604 are shown with baseline wander in the example 600, in variations, the ECG signal 602 and/or the PPG signal 604 may be baseline corrected.

The techniques described herein may leverage physiological information obtained from both the ECG signal 602 and the PPG signal 604 (e.g., the first physiological data 302 and the second physiological data 304, respectively) for time alignment purposes. Despite measuring different physiological phenomena, heart rate and pulse rate exhibit corresponding patterns over time. By way of example, a delay between the cardiac electrical activity captured by the ECG signal 602 and the peripheral pulse captured by the PPG signal 604 may reflect the pulse transit time from the heart to the peripheral measurement site. In one or more implementations, the synchronization algorithm 120 may analyze a time series of heart rate values derived from the RR interval 606, a time series of pulse rate values derived from the PP interval 608, and/or the pulse transit time to determine a timing reference (e.g., a time offset) for the monitoring device 104 and the auxiliary device 116. By way of example, the synchronization algorithm 120 may use cross-correlation or pattern matching techniques to identify corresponding patterns of heart rate and pulse rate changes between the two signals. By identifying when similar patterns of changes occur in both signals, the synchronization algorithm 120 may determine the temporal offset between device clocks and generate one or more synchronization signals to align data collection timing between the devices.

Alternatively, or in addition, the synchronization algorithm 120 may extract or infer respiratory information from both the ECG signal 602 and the PPG signal 604. The ECG signal 602, for instance, may be scaled or filtered to generate an ECG baseline modulation signal 610, which is shown as a dashed line following the baseline variations of the ECG signal 602. The ECG baseline modulation signal 610 may represent respiratory-induced variations such as respiratory sinus arrhythmia and amplitude modulation of the ECG signal 602, from which respiration rate may be derived. Similarly, the PPG signal 604 may be scaled or filtered to generate a PPG baseline modulation signal 612, which is shown as a dashed line following the baseline variations of the PPG signal 604. The PPG baseline modulation signal 612 may represent respiratory modulation effects on the PPG waveform through changes in venous return, intrathoracic pressure, and/or peripheral vascular resistance, from which the respiration rate may be derived. A time offset 614 is indicated between corresponding peaks (shown as open filled circles) and troughs (shown as black filled circles) in the ECG baseline modulation signal 610 and the PPG baseline modulation signal 612, with vertical dashed lines connecting the corresponding peaks and troughs to visually illustrate the temporal displacement. By cross-correlating the derived respiration signals from the ECG signal 602 and the PPG signal 604, the synchronization algorithm 120 may determine the time offset 614 between the monitoring device 104 and the auxiliary device 116. The time offset 614 represents the temporal misalignment between the two devices that can be corrected through generation of one or more synchronization signals as described herein. By way of example, the time offset 614 may provide the timing reference for generating the one or more synchronization signals. This approach enables accurate time alignment even when different types of sensors are used on different devices, leveraging common physiological features derived from different sensor types.

The following discussion describes techniques that are implementable utilizing the previously described systems and devices. Aspects of the procedure (e.g., method) can be implemented in hardware, firmware, software, or a combination thereof. The procedure is shown as a set of blocks that specify operations that can be performed by one or more devices and are not necessarily limited to the orders shown for performing the operations by the respective blocks. One or more blocks of the procedure, for instance, specify operations that can be programmable by hardware (e.g., a processor, microprocessor, controller, and/or firmware) as executable instructions, thereby creating a special purpose machine for carrying out an algorithm (e.g., the synchronization algorithm 120 of FIG. 1) as illustrated by the flow diagram. As a result, the instructions are storable on a computer-readable storage medium that causes the hardware to perform the algorithm. In portions of the following discussion, reference will be made to FIGS. 1-6.

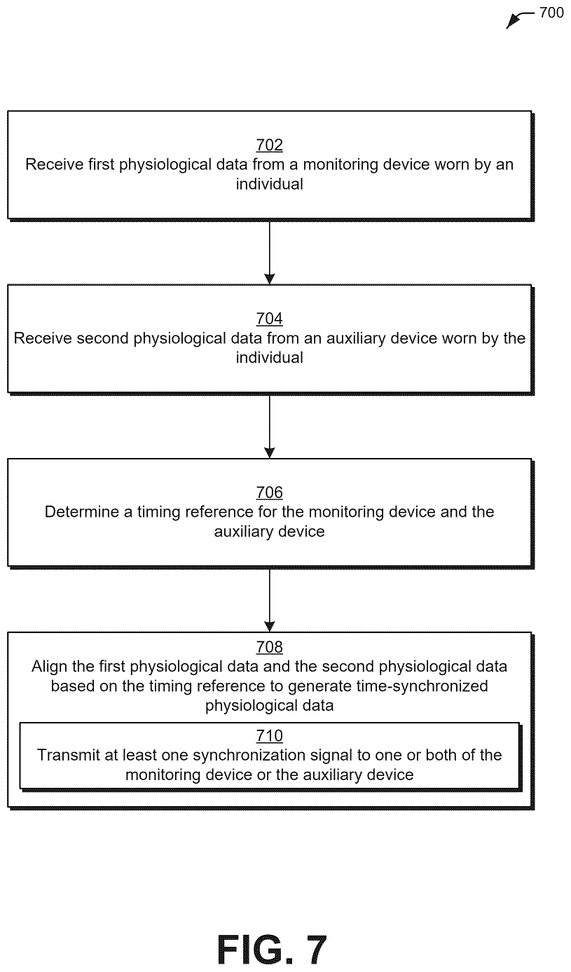

FIG. 7 illustrates a method 700 for time-aligning physiological data from multiple devices. The method 700 may be implemented by the analysis platform 106, the monitoring device 104, the auxiliary device 116, the separate primary device 502, and/or other suitable components of a physiological monitoring system. In at least one implementation, the method 700 is executed at least partially by a primary device, which may include or have access to the analysis platform 106 and the synchronization algorithm 120. In various implementations, the synchronization algorithm 120 may perform one or more operations of the method 700.

First physiological data are received from a monitoring device worn by an individual (block 702). By way of example, the first physiological data 302 may be received by the primary device, which may be the monitoring device 104 itself, the auxiliary device 116, or the separate primary device 502 such as a smartphone, gateway, or laptop. In implementations where the monitoring device 104 acts as the primary device, the monitoring device 104 may receive the first physiological data 302 from the one or more sensors 202 included therein. In implementations where the auxiliary device 116 or the separate primary device 502 acts as the primary device, the auxiliary device 116 or the separate primary device 502 receives the first physiological data from the monitoring device 104. In some implementations, the monitoring device 104 may be configured to be attached to the chest of the person 102. The first physiological data 302 may include a variety of measurement modalities. In one example, the monitoring device includes an electrocardiogram (ECG) sensor, and the first physiological data 302 include ECG data measuring electrical activity of the heart.

Second physiological data are received from an auxiliary device worn by the individual (block 704). As noted above, the primary device may be the monitoring device 104, the auxiliary device 116, or the separate primary device 502. In implementations where the auxiliary device 116 acts as the primary device, it may receive the second physiological data 304 from the one or more sensors 118 included therein. In implementations where the monitoring device 104 or the separate primary device 502 acts as the primary device, the monitoring device 104 or the separate primary device 502 receives the second physiological data 304 from the auxiliary device 116. The second physiological data 304 may include a variety of measurement modalities, which may include at least some of the same measurement modalities as the first physiological data 302. Alternatively, the second physiological data 304 may include different measurement modalities than the first physiological data 302.

In one or more implementations, the auxiliary device 116 may be configured to be worn by the person 102 at an extremity, such as a wrist or finger. In at least one variation, the auxiliary device 116 is attached to the neck of the person 102. For example, the auxiliary device 116 may be a smartwatch worn on a wrist, a finger ring device, or a neck-worn sensor. In one example, the auxiliary device 116 includes a photoplethysmogram (PPG) sensor, and the second physiological data 304 include PPG data and/or oxygen saturation measurements determined from the PPG data. In another example, the auxiliary device 116 includes a respiration sensor, and the second physiological data 304 include respiration data. In some implementations, the primary device may receive physiological data from multiple auxiliary devices simultaneously.

A timing reference for the monitoring device and the auxiliary device is determined (block 706). By way of example, determining the timing reference may include identifying a common physiological feature in the first physiological data 302 and the second physiological data 304. In one example, the common physiological feature may be a respiratory signal, where sensors of the monitoring device 104 and the auxiliary device 116 both capture time-varying respiratory signals, and the respiratory signals received from both devices can be utilized to determine a delay between the signals. The delay between the signals may be used as the timing reference. As described above with respect to FIG. 6, time-varying respiratory signals may be captured even when the monitoring device 104 and the auxiliary device 116 include different sensor modalities. By way of example, a respiration rate may be extracted from ECG signals through respiratory-induced variations such as respiratory sinus arrhythmia, baseline wander, and amplitude modulation of the ECG signal. Similarly, the respiration rate may be extracted from PPG signals through respiratory modulation effects on the PPG waveform. These derived respiration signals from ECG and PPG may be cross-correlated to determine a time offset between devices, which may be used as or may be used to determine the timing reference. The time offset may be calculated as a time difference between occurrences of the common physiological feature in the first physiological data and the second physiological data. The synchronization algorithm 120 may use cross-correlation or pattern matching techniques to identify corresponding features between the first physiological data 302 and the second physiological data 304.

Alternatively, or in addition, the timing reference may be determined for the monitoring device 104 and the auxiliary device 116 using wireless time reference information. By way of example, time alignment via wireless communication may use Bluetooth®, Wi-Fi®, or another wireless communication method between the monitoring device 104 and the auxiliary device 116 or between body-worn devices and the separate primary device 502 (e.g., a phone, gateway, or laptop). At least one of the monitoring device 104, the auxiliary device 116, or the separate primary device 502 may synchronize to a real-world time reference (e.g., global real-time). In such implementations, timing packets that embed absolute time referenced to the real-world time reference may be transmitted and recorded. By way of example, a given device may record the embedded absolute time together with a corresponding local receipt timestamp. The separate primary device 502, for instance, may obtain recorded timing events from the monitoring device 104 and the auxiliary device 116 and compute a clock offset by comparing the absolute time values and the local timestamps. Repeating this process during the observation period may provide multiple timing event pairs from which both an initial offset and a drift rate may be estimated. Alternatively, an initial timing event at the beginning of an applicable period (e.g., upon activation of the devices) may be used without repeated timing event measurements. The one or more timing events may describe the time offset between device clocks relative to the real-world time reference.

In yet another example, rather than the separate primary device 502, the monitoring device 104 or the auxiliary device 116 already includes or is connected to the real-world time reference and transmits the real-world time reference to the other device. By way of example, when the monitoring device 104 includes the real-world time reference, the monitoring device 104 may transmit the synchronization signal 306 including the real-world time reference to the auxiliary device 116. Alternatively, when the auxiliary device 116 includes the real-world time reference, the auxiliary device 116 may transmit the synchronization signal 306 including the real-world time reference to the monitoring device 104. In at least one variation, the monitoring device 104 or the auxiliary device 116 receives the real-world time reference from the separate primary device 502 and forwards the real-world time reference to the other of the monitoring device 104 or the auxiliary device 116.

The first physiological data and the second physiological data are aligned based on the timing reference to generate time-synchronized physiological data (block 708). By way of example, the alignment may be based in part on the timing reference determined through common physiological features, relationships between different physiological features, and/or using the real-world time reference, as described above. The alignment may include mapping, by the synchronization algorithm 120, timestamps of the first physiological data 302 and the second physiological data 304 to a common time axis using the determined timing reference. In some implementations, the synchronization algorithm 120 applies a constant offset, a time-varying offset that accounts for drift, or piecewise offsets derived from timing events recorded over time so that corresponding physiological events occur at the same time on the common time axis. The alignment may further include the synchronization algorithm 120 adjusting data to the corrected time points so that features such as ECG R-peaks in the first physiological data 302 and corresponding PPG pulses in the second physiological data 304 are aligned, for example. By way of example, the synchronization algorithm 120 may output time-corrected versions of the first physiological data 302 and the second physiological data 304 (e.g., having corrected timestamps) as well as other measurements acquired by the measurements 108 and/or the auxiliary device 116. The time-corrected measurements may enable accurate and efficient downstream processing by the analysis platform 106, including cross-modal correlation, event detection, and for generating the one or more predictions 110.

In at least one implementation, the aligning includes transmitting at least one synchronization signal to one or both of the monitoring device or the auxiliary device (block 710). The synchronization signal may include timing information that instructs devices (e.g., the monitoring device 104 and/or the auxiliary device 116) to adjust internal clocks or timestamp data relative to the timing reference (e.g., based on the real-world time reference and/or based on the time offset) so that newly acquired first physiological data 302 and second physiological data 304 are aligned. By way of example, real-time clocks within individual devices may maintain time-alignment during a wear time or a duration between time-syncing events based on the synchronization signal(s) transmitted by the primary device, which may be the monitoring device 104, the auxiliary device 116, or the separate primary device 502. In implementations using the separate primary device 502, the separate primary device 502 may emit synchronization signals to multiple body-worn devices.

In various examples, the synchronization algorithm 120 uses one or more machine learning models, e.g., to process various input data, determine the timing reference, generate the synchronization signal, and/or to time-align one or more devices as described herein. The alignment process performed by the method 700 (e.g., via the synchronization algorithm 120) may achieve temporal resolution that may be suitable for various applications.

By implementing the method 700, physiological data from and operations of multiple devices as well as the devices themselves may be accurately time-aligned, enabling precise temporal correlation between different physiological measurements. These time-synchronized data may support analysis of complex physiological events including multiple parameters and may improve diagnostic accuracy for conditions where timing relationships between physiological signals may be relevant. For example, the method enables accurate correlation of data from a chest-worn monitoring device with one or more auxiliary devices such as neck-worn respiration sensors and finger ring devices measuring SpO2 and/or PPG, supporting comprehensive assessment of conditions where temporal relationships between physiological parameters may be relevant.

### Machine Learning and AI in Time Alignment of Physiological Signals From Multiple Devices

The previous examples describe various instances of artificial intelligence (“AI”) models and/or machine learning models such as with respect to the synchronization algorithm 120 and/or the prediction system 114. In one or more examples, an AI model, e.g., a machine learning model, refers to a computer representation that is tunable (e.g., through training and retraining) based on inputs without being actively programmed by a user to approximate unknown functions, automatically and without user intervention. For instance, the term machine learning model includes a model that utilizes algorithms to learn from, and make predictions on, known data by analyzing training data to learn and relearn to generate outputs that reflect patterns and attributes of the training data.

In the context of time alignment of physiological signals from multiple devices, machine learning models are implementable (e.g., by one or more processing devices of the analysis platform 106) to analyze physiological data patterns such as to identify common physiological features, determine time offsets, and generate synchronization signals. For example, the synchronization algorithm 120 and/or the prediction system 114 may each utilize one or more machine learning models to process physiological data such as ECG readings, PPG signals, heart rate variability, respiratory patterns, and other measurements collected by the monitoring device 104 and the auxiliary device 116. Examples of machine learning models applicable to time alignment of physiological signals include neural networks, convolutional neural networks (CNNs) such as for analyzing waveform data and identifying corresponding features across different signal types, long short-term memory (LSTM) neural networks such as to analyze temporal physiological patterns and detect time-varying relationships between signals, generative adversarial networks (GANs), decision trees (e.g., for classification of synchronization quality), support vector machines, linear regression, logistic regression for binary synchronization success detection, Bayesian networks, random forest learning for feature importance in physiological signal alignment, dimensionality reduction algorithms, boosting algorithms, deep learning neural networks, and so forth.

A machine learning model, for instance, is configurable using a plurality of layers having, respectively, a plurality of nodes. The plurality of layers is configurable to include an input layer, an output layer, and one or more hidden layers. In the context of time alignment of physiological signals, the input layer may receive various physiological parameters from the measurements 108, such as ECG features, PPG waveforms, heart rate patterns, respiratory signals, motion data, and timing information from multiple devices. The hidden layers, for instance, process these inputs through weighted connections to identify complex patterns indicative of temporal relationships between physiological signals, e.g., patterns that are not detectable using conventional cross-correlation or peak detection methods. The output layer may produce time offset estimates, synchronization quality metrics, or generate the one or more predictions 110 that incorporate accurately time-aligned physiological data from multiple devices. Calculations are performed by the nodes within the layers via hidden states through a system of weighted connections that are “learned” during training of the machine learning model to implement a variety of time alignment and physiological assessment tasks.

In order to train the machine learning model for time alignment of physiological signals, training data are received that provide examples of “what is to be learned” by the machine learning model, i.e., as a basis to learn patterns from the data. For time alignment applications, the training data may include labeled datasets of physiological measurements from multiple devices with known time relationships, such as simultaneously recorded ECG and PPG signals with ground truth timing offsets, respiratory signals captured by different sensor types with verified synchronization, and/or physiological data from devices with artificially introduced time delays for training purposes. A machine learning system that includes the machine learning model, for instance, collects and preprocesses the training data that includes input features (e.g., ECG waveforms, PPG signals, respiratory patterns, heart rate intervals) and corresponding target labels (e.g., “time offset of 0.2 seconds,” “high synchronization confidence,” or specific temporal alignment classifications).

The machine learning system is further operable to initialize various parameters of the machine learning model, which are usable by the machine learning model as internal variables to represent and process information during training. These parameters are further usable to represent inferences gained through training. In one or more implementations, the training data are separated into batches to improve processing and optimization efficiency of the parameters of the machine learning model during training, which may be beneficial for model accuracy when processing large volumes of physiological time-series data from multiple devices with varying sampling rates and temporal characteristics.

The training data are then received by the machine learning model as inputs and used to generate predictions based on a current state of parameters of layers and corresponding nodes of the model, a result of which is output as output data, e.g., a time offset estimate, synchronization signal parameters, alignment quality assessment, or the like. For example, the analysis platform 106 includes a machine learning model that is trained to recognize patterns in physiological data that correlate with temporal relationships between signals from different devices, which enables the synchronization algorithm 120 to generate accurate time offset determinations and the prediction system 114 to process time-synchronized physiological data for improved diagnostic accuracy.

Training of the machine learning model can include calculation of a loss function to quantify a loss associated with operations performed by nodes of the machine learning model. The loss function is configurable in various ways to control operation and/or functionality of the machine learning model. For instance, the loss function may be designed to prioritize accuracy in time offset estimation while minimizing synchronization errors that could lead to misaligned physiological events and incorrect diagnostic conclusions. Calculation of the loss function, for instance, includes comparing a difference between predictions specified in the output data (e.g., predicted time offsets or synchronization quality metrics) with target labels specified by the training data (e.g., verified ground truth timing relationships). The loss function is configurable in a variety of ways, examples of which include regret, Quadratic loss function as part of a least squares technique for continuous time offset parameters, cross-entropy loss for synchronization quality classification tasks, custom loss functions that incorporate temporal precision requirements specific to particular physiological monitoring applications, and so forth.

The training data are usable to support a variety of usage scenarios in time alignment of physiological signals. For example, the machine learning model can be trained to detect specific patterns in physiological data (e.g., ECG and PPG data) that enable accurate heart rate and pulse rate correlation, identify respiratory patterns derived from different sensor modalities for cross-correlation analysis, recognize motion artifacts that may affect synchronization accuracy, or detect subtle temporal relationships between physiological parameters that may improve alignment precision. The models can be configured to operate within computational constraints of real-time synchronization while providing accurate time offset estimates. The models can further be reconfigured, e.g., with expanded capabilities, for more sophisticated temporal analysis when processing historical data or performing detailed diagnostic assessments. This adaptive approach enables efficient use of computational resources devoted to machine learning processes while ensuring comprehensive time alignment capabilities are available when needed for accurate correlation of physiological events across multiple devices and measurement modalities.

It should be understood that many variations are possible based on the disclosure herein. Although features and elements are described above in particular combinations, each feature or element is usable alone without the other features and elements or in various combinations with or without other features and elements.

What is claimed is:

1. A method for time-alignment between physiological signals from a monitoring device and an auxiliary device, comprising:
receiving, by a primary device, first physiological data from the monitoring device worn by an individual;
receiving, by the primary device, second physiological data from the auxiliary device worn by the individual;
determining a timing reference for the monitoring device and the auxiliary device; and
aligning the first physiological data and the second physiological data based on the timing reference to generate time-synchronized physiological data.

2. The method of claim 1, wherein aligning the first physiological data and the second physiological data based on the timing reference includes generating, by the primary device, a synchronization signal for transmission to at least one of the monitoring device or the auxiliary device based on the timing reference.

3. The method of claim 2, wherein the timing reference is a real-world time reference, and wherein the synchronization signal includes timing information that instructs at least one of the monitoring device or the auxiliary device to adjust an internal clock relative to the real-world time reference.

4. The method of claim 2, further comprising:
recording, by at least one of the monitoring device or the auxiliary device, timing information included in the synchronization signal relative to internal timing information.

5. The method of claim 1, wherein the monitoring device is the primary device, and the auxiliary device is a secondary device.

6. The method of claim 1, wherein the auxiliary device is the primary device, and the monitoring device is a secondary device.

7. The method of claim 1, wherein the primary device is an additional auxiliary device.

8. The method of claim 1, wherein determining the timing reference comprises:
identifying a common physiological feature in the first physiological data and the second physiological data; and
calculating, as the timing reference, a time difference between occurrences of the common physiological feature in the first physiological data and the second physiological data.

9. The method of claim 1, further comprising:
identifying a first physiological feature in the first physiological data;
identifying a second physiological feature in the second physiological data; and
determining the timing reference based at least in part on a relationship between the first physiological feature and the second physiological feature.

10. The method of claim 9, wherein the first physiological feature is heart rate, and the second physiological feature is pulse rate.

11. The method of claim 9, wherein:
the monitoring device is configured to be attached to a chest of the individual and includes an electrocardiogram (ECG) sensor for measuring, as the first physiological data, ECG data; and
the auxiliary device is configured to be attached to an extremity of the individual and includes a photoplethysmogram (PPG) sensor for measuring, as the second physiological data, PPG data.

12. The method of claim 11, wherein the timing reference is based at least in part on respiratory signals extracted from the ECG data and the PPG data.

13. A system for physiological monitoring of an individual, comprising:
a monitoring device configured to measure first physiological data at a first body location of the individual;
an auxiliary device configured to measure second physiological data at a second body location of the individual; and
a synchronization algorithm implemented as computer-readable instructions stored in a non-transitory memory that, when executed by a processing device, cause the processing device to perform operations comprising:
determining a timing reference for the monitoring device and the auxiliary device; and
time-aligning the first physiological data and the second physiological data based on the timing reference to generate time-synchronized physiological data.

14. The system of claim 13, wherein the monitoring device or the auxiliary device comprises the processing device.

15. The system of claim 13, further comprising the processing device, and wherein the processing device is a smartphone, gateway or laptop.

16. The system of claim 13, wherein to determine the timing reference, the operations further comprise:
identifying a common physiological feature in the first physiological data and the second physiological data; and
calculating, as the timing reference, a time difference between occurrences of the common physiological feature in the first physiological data and the second physiological data.

17. The system of claim 13, wherein to determine the timing reference, the operations further comprise:
transmitting timing information corresponding to a real-world time to at least one of the monitoring device or the auxiliary device;
receiving recorded timing events from at least one of the monitoring device and the auxiliary device, each recorded timing event including the timing information and a corresponding local timestamp; and
determining the timing reference based on the timing information and the corresponding local timestamp.

18. A method for time-alignment between physiological signals from multiple wearable devices, comprising:
measuring, by a monitoring device, electrocardiogram (ECG) data at a first body location of an individual;
measuring, by an auxiliary device, photoplethysmogram (PPG) data at a second body location of the individual;
transmitting, by a primary device, a synchronization signal including a timing reference to at least one of the monitoring device or the auxiliary device; and
time-aligning, by the primary device, the ECG data and the PPG data based on the timing reference.

19. The method of claim 18, further comprising:
identifying at least one physiological feature in the ECG data and the PPG data; and
determining the timing reference based on a time difference between occurrences of the at least one physiological feature in the ECG data and the PPG data.

20. The method of claim 18, wherein the timing reference corresponds to a real-world time.
